(19) United States
(12) Patent Application Publication
(10) Pub. No.: US 2026/0080492 A1
(43) Pub. Date: Mar. 19, 2026

(54) SYSTEMS AND METHODS FOR VEHICLE EVENTS AND REAL-TIME INTERACTIVE ASSISTANCE

(71) Applicant: Toyota Motor North America, Inc., Plano, TX (US)

(72) Inventors: Simon P. Roberts, Celina, TX (US); Dineth Kumarasinghe, Flower Mound, TX (US); William Hargis, Celina, TX (US); David Tsai, Irvine, CA (US); Shravanthi Denthumdas, Frisco, TX (US); Brian Kursar, Fairview, TX (US); Robert Heckel, Katy, TX (US); Mark A. McClung, Celina, TX (US); Steven S. Basra, Frisco, TX (US); Charan S. Lota, Frisco, TX (US); Benjamin R. Resnick, Plano, TX (US); Ryan Wheeler, Denton, TX (US); Lokesh Kumar Viswavarapu, Frisco, TX (US); Raja Shekar Kilaru, Addison, TX (US); Nikhil Rajendra, Plano, TX (US)

(73) Assignees: Toyota Motor North America, Inc., Plano, TX (US); Toyota Jidosha Kabushiki Kaisha, Aichi-ken (JP)

(21) Appl. No.: 18/885,040

(22) Filed: Sep. 13, 2024

(51) Int. Cl. G06Q 50/26; G06V 20/58; G16H 50/20; H04M 3/51; H04N 7/18

(52) U.S. Cl. CPC G06Q 50/265; G06V 20/58; G16H 50/20; H04M 3/5116; H04N 7/183

(57) ABSTRACT

A vehicle event system includes a processor and a non-transitory, processor-readable storage medium communicatively coupled to the processor, the non-transitory, processor-readable storage medium including one or more instructions stored thereon that, when executed, cause the processor to obtain sensor data and image acquisition data; generate a visualization of an event of a vehicle based on the sensor data and the image acquisition data; display, on a processing device, the visualization of the event; and transmit, via an accident assistant module, audio data to one or more passengers of the vehicle for real-time interactive assistance.

### FIELD

The present disclosure generally relates to devices for events, and more particularly, to vehicle events and real-time interactive assistance.

### BACKGROUND

Vehicles, and information exchanged therefrom, play an integral role in the well-being of users. However, there are numerous challenges in obtaining and utilizing information surrounding specifics of incidents involving a vehicle. These and other deficiencies exist.

### BRIEF DESCRIPTION OF THE DRAWINGS

In one aspect, a vehicle event system may include a processor, and a non-transitory, processor-readable storage medium communicatively coupled to the processor, the non-transitory, processor-readable storage medium comprising one or more instructions stored thereon that, when executed, cause the processor to: obtain sensor data and image acquisition data; generate a visualization of an event of a vehicle based on the sensor data and the image acquisition data; display, on a processing device, the visualization of the event; and transmit, via an accident assistant module, audio data to one or more passengers of the vehicle for real-time interactive assistance.

In another aspect, a method may include obtaining sensor data and image acquisition data. The method may include generating a visualization of an event of a vehicle based on the sensor data and the image acquisition data. The method may include displaying, on a processing device, the visualization of the event. The method may include transmitting, via an accident assistant module, audio data to one or more passengers of the vehicle for real-time interactive assistance.

In another aspect, a non-transitory, computer-readable medium including instructions that, when executed by at least one processor, cause the at least one processor to perform one or more operations including obtaining sensor data and image acquisition data; generating a visualization of an event of a vehicle based on the sensor data and the image acquisition data; displaying, on a processing device, the visualization of the event; and transmitting, via an accident assistant module, audio data to one or more passengers of the vehicle for real-time interactive assistance.

These and other features, and characteristics of the present technology, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure. As used in the specification and in the claims, the singular form of ‘a’, ‘an’, and ‘the’ include plural referents unless the context clearly dictates otherwise.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, wherein like structure is indicated with like reference numerals and in which:

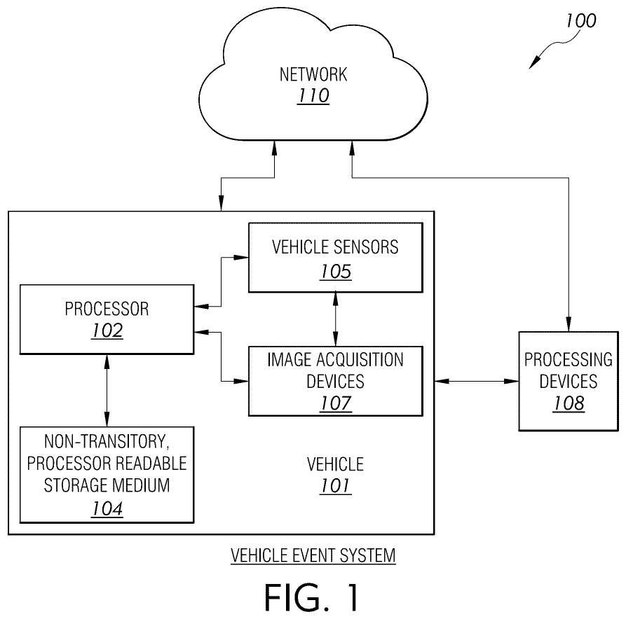

FIG. 1 depicts a schematic diagram of an example vehicle event system, according to one or more embodiments shown and described herein;

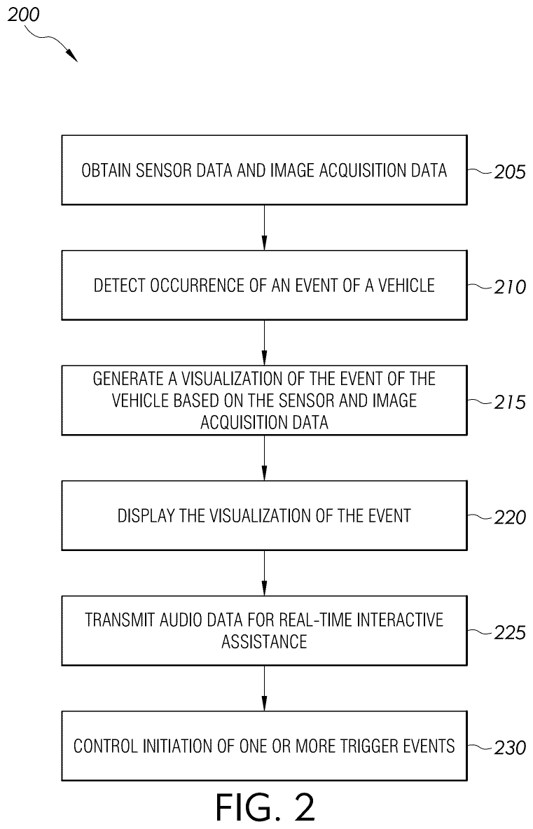

FIG. 2 depicts a flow diagram of an example method to be performed by a processor, according to one or more embodiments shown and described herein; and

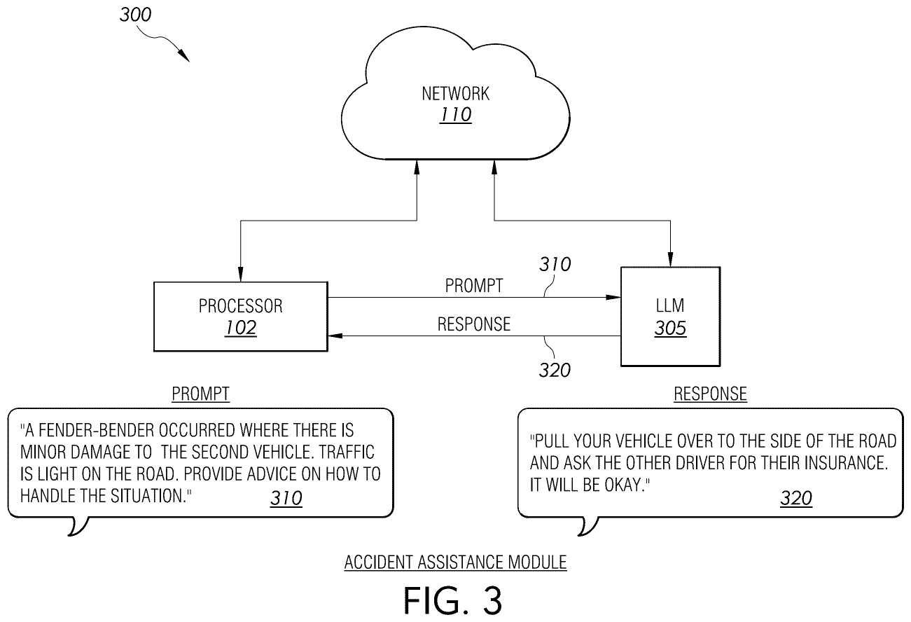

FIG. 3 depicts a schematic diagram of an example machine learning model prompt, according to one or more embodiments shown and described herein.

### DETAILED DESCRIPTION

The present disclosure relates to systems and methods for vehicle events and real-time interactive assistance. Utilizing generative artificial intelligence, the systems and methods disclosed herein may be configured to generate a visualization and summary of an event, such as a vehicle accident, based on on-board sensor data and cameras, and also generate an accident assistant that speaks to a driver of the vehicle through an audio system. The generative artificial intelligence systems and methods disclosed herein may be used for personal records, for an insurance company, and/or for a government authority. To reproduce and display a visualization of the accident scene, the systems and methods disclosed herein may not only obtain data from sensors and cameras onboard a vehicle, but also from other data sources, such as the Internet of Things, in order to accurately capture the full scope of the accident. The systems and methods disclosed herein not only reproduce and the display the visualization of the accident scene, but further, the systems and methods disclosed herein interact, in real-time, with a driver or passenger of the vehicle as part of interactive assistance to determine next steps immediately following the accident, thereby improving upon user interface experience. By utilizing generative artificial intelligence to reproduce an accident scene, a realistic and informative visualization of the accident may be provided by the systems and methods disclosed herein. The generative AI model can synthesize vehicle trajectories, accurately depicting their positions, velocities, and interactions leading up to the collision. This enables the generative AI model to generate a visually compelling and informative representation of the accident, aiding in accident analysis, reconstruction, and understanding of the event. Further, the collected sensor data provides assistance to the driver in real-time in coping with the accident in an instructive and collaborative manner.

FIG. 1 depicts a schematic diagram of an example vehicle event system 100. As illustrated in FIG. 1, the vehicle event system 100 includes a vehicle 101, a processor 102, a non-transitory processor readable storage medium 104, on-board vehicle sensors 105, on-board vehicle image acquisition devices 107, processing devices 108, and a network 110. Although FIG. 1 illustrates single instances of the constituent components of the vehicle event system 100, the vehicle event system 100 may include any number of constituent components.

In certain embodiments, the vehicle 101 may include an autonomous driving vehicle. In other embodiments, the vehicle 101 may include a vehicle that is not an autonomous driving vehicle. Without limitation, the vehicle may include a passenger vehicle, a non-passenger vehicle, a taxi, a bus, a scooter, a motorcycle, a truck, or any other type of vehicle.

The processor 102, such as a central processing unit (CPU), may be the central processing unit that is configured to perform calculations and logic operations to execute one or more programs. The processor 102, alone or in conjunction with the other components, may be an illustrative processing device, computing device, processor, or combinations thereof, including, for example, a multi-core processor, a microcontroller, a field-programmable gate array (FPGA), or an application-specific integrated circuit (ASIC). The processor 102 may include any processing component configured to receive and execute instructions (such as from the non-transitory processor readable storage medium 104).

In some examples, the processing device may execute one or more applications, such as software applications, that enable, for example, network communications with one or more components of the system 100 and transmit and/or receive data.

The non-transitory processor readable storage medium 104 may contain one or more data repositories for storing data that is received and/or generated. The non-transitory processor readable storage medium 104 may be any physical storage medium, including, but not limited to, a hard disk drive (HDD), memory (e.g., read-only memory (ROM), programmable read-only memory (PROM), random access memory (RAM), double data rate (DDR) RAM, flash memory, and/or the like), removable storage, a configuration file (e.g., text) and/or the like. While the non-transitory processor readable storage medium 104 is depicted as a local device, it should be understood that the non-transitory processor readable storage medium 104 may be a remote storage device, such as, for example, a server computing device, cloud-based storage device, or the like.

The on-board vehicle sensors 105 may include one or more on-board vehicle sensors. By way of example, the on-board vehicle sensors may include any number and type of vehicle sensors that may be each configured to obtain sensor data pertaining to an event. Without limitation, these vehicle sensors may include speed sensors, acceleration sensors, brake sensors, pressure sensors, impact sensors, temperature sensors, fuel sensors, tire sensors, engine sensors, error sensors, or any combination thereof.

The on-board vehicle image acquisition devices 107 may include one or more on-board vehicle image acquisition devices. By way of example, the one or more on-board vehicle image acquisition devices may include any number and type of vehicle cameras that may be each configured to obtain the image acquisition data and/or video data pertaining to an event.

The processing devices 108 may include one or more processing devices. By way of example, the processing device 108 may be a network-enabled computer. As referred to herein, a network-enabled computer may include, but is not limited to a computer device, or communications device including, e.g., a server, a network appliance, a personal computer, a workstation, a phone, a handheld PC, a personal digital assistant, a thin client, a fat client, an Internet browser, or other device. The processing device 108 also may be a mobile device; for example, a mobile device may include an iPhone, iPod, iPad from Apple® or any other mobile device running Apple's iOS® operating system, any device running Microsoft's Windows® Mobile operating system, any device running Google's Android® operating system, and/or any other smartphone, tablet, or like wearable mobile device.

The processing device 108 can include a processor and a memory, similar or different than processor 102 and non-transitory processor readable storage medium 104, and it is understood that the processing circuitry may contain additional components, including processors, memories, error and parity/CRC checkers, data encoders, anticollision algorithms, controllers, command decoders, security primitives and tamperproofing hardware, as necessary to perform the functions described herein. The processing device 108 may further include a display and input devices. The display may be any type of device for presenting visual information such as a computer monitor, a flat panel display, and a mobile device screen, including liquid crystal displays, light-emitting diode displays, plasma panels, and cathode ray tube displays. The input devices may include any device for entering information into the user's device that is available and supported by the user's device, such as a touch-screen, keyboard, mouse, cursor-control device, touch-screen, microphone, digital camera, video recorder or camcorder. These devices may be used to enter information and interact with the software and other devices described herein.

The network 110 may be one or more of a wireless network, a wired network, or any combination of wireless network and wired network, and may be configured to operably communicate with any and all of the constituent components of the vehicle event system 100. For example, network 110 may include one or more of a fiber optics network, a passive optical network, a cable network, an Internet network, a satellite network, a wireless local area network (LAN), a Global System for Mobile Communication, a Personal Communication Service, a Personal Area Network, Wireless Application Protocol, Multimedia Messaging Service, Enhanced Messaging Service, Short Message Service, Time Division Multiplexing based systems, Code Division Multiple Access based systems, D-AMPS, Wi-Fi, Fixed Wireless Data, IEEE 802.11b, 802.15.1, 802.11n and 802.11g, Bluetooth, NFC, Radio Frequency Identification (RFID), Wi-Fi, and/or the like. In addition, the network 110 may include, without limitation, telephone lines, fiber optics, IEEE Ethernet 802.3, a wide area network, a wireless personal area network, a LAN, or a global network such as the Internet. In addition, the network 110 may support an Internet network, a wireless communication network, a cellular network, or the like, or any combination thereof. The network 110 may further include one network, or any number of the exemplary types of networks mentioned above, operating as a stand-alone network or in cooperation with each other. The network 110 may utilize one or more protocols of one or more network elements to which they are communicatively coupled. The network 110 may translate to or from other protocols to one or more protocols of network devices. Although the network 110 is depicted as a single network, it should be appreciated that in one or more aspects, the network 110 may include a plurality of interconnected networks, such as, for example, the Internet, a service provider's network, a cable television network, corporate networks, such as credit card association networks, and home networks.

The processor 102 may be configured to obtain sensor data and image acquisition data. The processor 102 may be configured to obtain the sensor data from one or more on-board vehicle sensors 105. Without limitation, the sensor data may include surrounding external environment sensor data, relative to the vehicle 101, that may be recorded as a function of time. Further, the processor 102 may be configured to obtain the image acquisition data from one or more on-board vehicle image acquisition devices 107. In certain embodiments, the one or more on-board vehicle sensors 105 and/or the one or more on-board vehicle image acquisition devices 107 may be located not only within the vehicle 101 but also exterior to the vehicle 101.

The processor 102 may be configured to detect an occurrence of the event of the vehicle 101 based on the one or more on-board vehicle sensors 105 and the one or more on-board vehicle image acquisition devices 107. In certain embodiments, the event may include an accident of the vehicle 101. Without limitation, the accident of the vehicle 101 may or may not be relative to a second vehicle that is parked or moving. In other examples, the accident of the vehicle 101 may be relative to an event not caused by a second vehicle, such as the vehicle 101 losing vehicle control and striking, for example, a curb, a road sign, a grocery cart, a pillar, or any other object. In other examples, the vehicle 101 may lose vehicle control, such as complete or partial autonomous vehicle control due to inclement weather and/or traffic conditions and/or vehicle equipment malfunction and/or sudden object appearance relative to the surrounding of the vehicle 101.

The processor 102 may be configured to generate a visualization of the event of the vehicle 101 based on the sensor data and the image acquisition data. For example, the visualization of the event of the vehicle 101 may include visually reproducing, such as in a media format of obtained images and/or videos, the accident. In this manner, a realistic and informative visualization to reproduce an accident scene involving the vehicle 101 may be generated and provided by the processor.

In certain embodiments, visualization of the event may include vehicle trajectory data, collision dynamics data, environment data, traffic data, accident sequence data over a predetermined time period, object data, landmark data, or any combination thereof. Additionally or alternatively, visualization of the event may include angle data and direction data relative to a respective time period as pertaining to the event. Additionally or alternatively, visualization of the event may include video data, image data, vehicle impact data, accident information data, or any combination thereof.

In certain embodiments, the visualization of the event may be generated by the processor 102, that is also configured to obtain data from any number of other data sources, including but not limited to Internet of Things (IoT), to further refine and generate the visualization of the event. By way of example, the processor 102 may be configured to compare the event against the actual accident scene as captured by IoT. For example, the processor 102 may be configured to determine that the visualization of the event that it is able to generate on its own does not represent the full scope of the accident. Accordingly, the processor 102 may be configured to obtain relevant accident data from other sensors from the IoT, that itself captured the actual accident, to reproduce the visualization of the event of the vehicle 101 in a complete form.

The processor 102 may be configured to display, on a processing device 108, the visualization of the event. For example, the processor 102 may be configured to display the visualization of the event on a display of vehicle 101, including but not limited to a dashboard monitor display of the vehicle 101. In other example, the processor 102 may be configured to display the visualization of the sevent on a display of a processing device 108 of the driver of the vehicle 101. Still further, the processor 102 may be configured to display the visualization of the event on a display of a processing device 108 of another user of the vehicle 101. Still further, the processor 102 may be configured to display the visualization of the event on a display of a processing device 108 of a different driver of another vehicle. Still further, the processor 102 may be configured to display the visualization of the event on a display of a processing device 108 located external or remote relative to the vehicle 101, including but not limited to a server, a database, or any other type of device. In some examples, the processor 102 may be configured to upload the visualization of the event to the server, the database, the device, and/or the cloud for subsequent display. It is understood that the processor 102 may be configured to validate the generated visualization prior to transmitting it for display.

The processor 102 may be configured to transmit, via an accident assistant module, audio data to one or more passengers of the vehicle 101 for real-time interactive assistance. The processor 102 may be further configured to generate, after detection of the occurrence of the event, the accident assistant module using generative artificial intelligence, such as a neural network, as described with reference to FIG. 3.

The accident assistant module may be trained by the one or more on-board vehicle sensors 105 and/or the one or more on-board vehicle image acquisition devices 107. In certain embodiments, the accident assistant module may be configured to transmit the audio data to a driver of the vehicle 101. For example, the audio data may be transmitted by the accident assistant module via an audio system of the vehicle 101. It is understood that the transmission of the audio data for the real-time interactive assistance is not limited to only the audio system of the vehicle 101, and that other audio systems may be used that are not part of the vehicle 101, such as the audio system of the processing device 108 of the driver. In certain embodiments, the accident assistant module may be configured to receive, in response to the transmitted audio data, responsive data from the driver of the vehicle 101. In certain embodiments, the accident assistant module may be configured to control initiation, in response to the responsive data, of one or more trigger events. By way of example, the responsive data may be received and recognized by the accident assistant module via the audio system of the vehicle 101. In another example, the responsive data may be received and recognized by the accident assistant module via the audio system of the processing device of the driver. Without limitation, the responsive data may include audio data.

In certain embodiments, the one or more trigger events may include providing instructions to guide the vehicle 101, requesting information from a second driver, or any combination thereof. For example, the real-time interactive assistance may be associated with any number of the one or more trigger events. Without limitation, providing instructions to guide the vehicle may include automatically maneuvering the vehicle 101 to pull it to the side of the road, such as in the case of an autonomous driving vehicle, and/or instructing the driver to pull the vehicle 101 to the side of the road. Other examples of instructions provided include driving the vehicle 101 at a predetermined speed limit, initiating vehicle brakes, and/or steering the vehicle 101 in a predetermined direction. It is understood that these instructions may be provided to an autonomous driving vehicle, which in response is configured to carry out or otherwise perform the given instructions. In other examples, it is understood that these instructions may be provided to a vehicle that is not an autonomous driving vehicle.

Without limitation, requesting information from a second driver may include requesting insurance card information from the second driver. The second driver may correspond to the driver associated with the event, such as the vehicle accident, by a second vehicle. In certain embodiments, the processor 102 may be configured to determine, based on the insurance card information, whether the insurance of the second driver has expired. For example, the processor 102 may be configured to receive the insurance card information from a mobile device associated with a driver of the second vehicle. Of course, it is understood that the processor 102 is not limited to receiving the insurance card information in this manner, and that other techniques to obtain or receive this information be utilized, including but not limited to connecting to a server or a database that hosts this information, transmitting this information through a phone gesture from the mobile device associated with the driver of the second vehicle, such as a tap or a wave to any constituent component of system 100, or through wireless communication RFID, NFC, Bluetooth, email, short message service and/or wired, such as a USB cable, communication. To the extent that the processor determines that the insurance of the second driver has expired, the processor 102 may be further configured to take corrective action. By way of example, the corrective action may include automatically initiating and establishing communication with an emergency dispatcher and/or police and/or fire station and/or hospital. In certain embodiments, the corrective action performed by the processor 102 may be based on, for example, a location of the vehicle 101 and the second vehicle engaged in the event, and thereby determine the nearest police station, fire station, and/or hospital to automatically initiate and establish communication thereto.

It is understood that the embodiments described above is not limited to controlling initiating and establishing, by the processor 102, communication with a particular emergency dispatcher, or limited to only a second driver, or limited to only a second vehicle. Rather, any number and types of emergency dispatchers as well as any number of drivers and vehicles may be included. Further, it is understood that the drivers are not limited thereto, and that any other number and any other types of passengers may be included.

The processor 102 may be configured to determine one or more degrees of the event of the vehicle 101. Without limitation, the processor may be configured to determine a first degree of the event of the vehicle 101 and also a second degree of the event of the vehicle 101. In certain embodiments, the processor 102 may be configured to determine a first degree of the event. For example, the first degree of the event may include a minor accident associated with the vehicle 101. Without limitation, the minor accident may include a fender bender, a cracked windshield, a busted tire, a small damaged portion of an exterior of the vehicle, or any combination thereof. The minor accident may or may not be due to another vehicle. In other examples, the processor 102 may be configured to determine a second degree of the event of the vehicle 101. Further, the processor 102 may be configured to determine, based on the event comprising the minor accident, whether the vehicle 101 is driveable. For example, the processor 102 may be configured to determine, after determining the first degree of the event of the vehicle, a type of injury using one or more on-board vehicle image acquisition devices 107. By way of example, the one or more on-board vehicle image acquisition devices 107 may include any number of the interior and/or exterior cameras that may be each configured to obtain the image acquisition data and video data pertaining to the event, and in particular, the minor accident. The type of injury may include, but not be limited to, a foot injury, a head injury, or any other bodily injury relative to the driver and/or passengers of the vehicle 101. The bodily injury associated with the minor accident may be minor as compared to that of the bodily injury associated with the major accident, as further explained below.

In other examples, the processor 102 may be configured to determine if a claim, such as an insurance claim, can be made. For example, the processor 102 may be configured to establish communication with drivers and/or users of another vehicle, and thereafter receive and process payment between the parties of the vehicle and the parties of another vehicle, including exchanging information between these parties without the need for physical interaction between the parties. By way of example, the processor 102 may be configured to establish communication with a user device of the another vehicle, and thereby transmit and receive the exchanged information sufficient to process filing of a claim and/or payment between the parties. The processor 102 may be configured to determine if the insurance of another driver from another vehicle has expired and recommend processing of an insurance claim that takes into account such a consideration. In certain embodiments, the processor may 102 may be configured to obtain an adjuster, such as in-person or to automatically perform one or more of the following operations via a video session (which may be pre-recorded on in real-time) and/or a camera session: record the image data and video data of the vehicle(s), assess the image data and the video data relative to damage caused to the vehicle(s) due to the event as well as the scope of any injurie(s) to any drivers and/or passengers, produce an estimate of an amount of vehicle(s) damage(s) and injuri(es), and determine whether the estimate exceeds a threshold so as to determine if the insurance claim can be made.

Upon determining the type of injury using the one or more on-board vehicle image acquisition devices 107, the processor 102 may be further configured to initiate and establish communication with a call center associated with the emergency dispatcher and/or the police and/or the fire station and/or the hospital.

In certain embodiments, the second degree of the event may include a major accident associated with the vehicle 101. By way of example, the major accident may be more serious and significant as compared to the minor accident of the vehicle 101. For example, the major accident may result in a substantial or totaling of the vehicle 101 and/or major bodily damage to any passengers or drivers of any number of vehicles.

The processor 102 may be configured to generate a summary of the event of the vehicle 101 based on the sensor data and the image acquisition data. For example, the summary of the event may include any number and types of reports, such as a tabularized version report, to provide vehicle trajectory data, vehicle speed data, time of day data, video data, image data, and vehicle impact data relative to time periods preceding the event, during the event, and after the event.

In certain embodiments, the driver may provide feedback to continuously improve and train the accident assistant module. For example, the driver may provide feedback, either via a request prompt or proactively, to correct or modify the instructions that it receives from the accident assistant module via the audio system. In this manner, the accident assistant module may be configured to receive the feedback, such as via the audio system or a processing device of the driver, and incorporate it to improve its instructions that it provides.

FIG. 2 depicts a flow diagram of an example method 200 performed by the processor 102. FIG. 2 may reference and incorporate any of the above constituent components and corresponding disclosure explained above with respect to FIG. 1, such as the example vehicle event system 100.

At block 205, the processor 102 obtains sensor data and image acquisition data. The processor 102 may be configured to obtain the sensor data from one or more on-board vehicle sensors 105. Without limitation, the sensor data may include surrounding external environment sensor data, relative to the vehicle 101, that may be recorded as a function of time. Further, the processor 102 may be configured to obtain the image acquisition data from one or more on-board vehicle image acquisition devices 107. In certain embodiments, the one or more on-board vehicle sensors 105 and/or the one or more on-board vehicle image acquisition devices 107 may be located not only within the vehicle 101 but also exterior to the vehicle 101.

At block 210, the processor 102 detects an occurrence of an event of the vehicle. The processor 102 may be configured to detect an occurrence of the event of the vehicle 101 based on the one or more on-board vehicle sensors 105 and the one or more on-board vehicle image acquisition devices 107. In certain embodiments, the event may include an accident of the vehicle 101. Without limitation, the accident of the vehicle 101 may or may not be relative to a second vehicle that is parked or moving. In other examples, the accident of the vehicle 101 may be relative to an event not caused by a second vehicle, such as the vehicle 101 losing vehicle control and striking, for example, a curb, a road sign, a grocery cart, a pillar, or any other object. In other examples, the vehicle 101 may lose vehicle control, such as complete or partial autonomous vehicle control due to inclement weather and/or traffic conditions and/or vehicle equipment malfunction and/or sudden object appearance relative to the surrounding of the vehicle 101.

At block 215, the processor 102 generates a visualization of the event of the vehicle based on the sensor and image acquisition data. The processor 102 may be configured to generate a visualization of the event of the vehicle 101 based on the sensor data and the image acquisition data. For example, the visualization of the event of the vehicle 101 may include visually reproducing, such as in a media format of obtained images and/or videos, the accident. In this manner, a realistic and informative visualization to reproduce an accident scene involving the vehicle 101 may be generated and provided by the processor 102.

In certain embodiments, visualization of the event may include vehicle trajectory data, collision dynamics data, environment data, traffic data, accident sequence data over a predetermined time period, object data, landmark data, or any combination thereof. Additionally or alternatively, visualization of the event may include angle data and direction data relative to a respective time period as pertaining to the event. Additionally or alternatively, visualization of the event may include video data, image data, vehicle impact data, accident information data, or any combination thereof.

In certain embodiments, the visualization of the event may be generated by the processor 102, that is also configured to obtain data from any number of other data sources, including but not limited to Internet of Things (IoT), to further refine and generate the visualization of the event. By way of example, the processor 102 may be configured to compare the event against the actual accident scene as captured by IoT. For example, the processor 102 may be configured to determine that the visualization of the event that it is able to generate on its own does not represent the full scope of the accident. Accordingly, the processor 102 may be configured to obtain relevant accident data from other sensors from the IoT, that itself captured the actual accident, to reproduce the visualization of the event of the vehicle 101 in a complete form.

At block 220, the processor 102 displays the visualization of the event. The processor 102 may be configured to display, on a processing device 108, the visualization of the event. For example, the processor 102 may be configured to display the visualization of the event on a display of vehicle 101, including but not limited to a dashboard monitor display of the vehicle 101. In other example, the processor 102 may be configured to display the visualization of the event on a display of a processing device 108 of the driver of the vehicle 101. Still further, the processor 102 may be configured to display the visualization of the event on a display of a processing device 108 of another user of the vehicle 101. Still further, the processor 102 may be configured to display the visualization of the event on a display of a processing device 108 of a different driver of another vehicle. Still further, the processor 102 may be configured to display the visualization of the event on a display of a processing device 108 located external or remote relative to the vehicle 101, including but not limited to a server, a database, or any other type of device. In some examples, the processor 102 may be configured to upload the visualization of the event to the server, the database, the device, and/or the cloud for subsequent display. It is understood that the processor 102 may be configured to validate the generated visualization prior to transmitting it for display.

At block 225, the processor transmits audio data for real-time interactive assistance. The processor 102 may be configured to transmit, via an accident assistant module, audio data to one or more passengers of the vehicle 101 for real-time interactive assistance. The processor 102 may be further configured to generate, after detection of the occurrence of the event, the accident assistant module using generative artificial intelligence, such as a neural network. The accident assistant module may be trained by the one or more on-board vehicle sensors 105 and/or the one or more on-board vehicle image acquisition devices 107. In certain embodiments, the accident assistant module may be configured to transmit the audio data to a driver of the vehicle 101. For example, the audio data may be transmitted by the accident assistant module via an audio system of the vehicle 101. It is understood that the transmission of the audio data for the real-time interactive assistance is not limited to only the audio system of the vehicle 101, and that other audio systems may be used that are not part of the vehicle 101, such as the audio system of the processing device 108 of the driver. In certain embodiments, the accident assistant module may be configured to receive, in response to the transmitted audio data, responsive data from the driver of the vehicle 101. In certain embodiments, the accident assistant module may be configured to control initiation, in response to the responsive data, of one or more trigger events. By way of example, the responsive data may be received and recognized by the accident assistant module via the audio system of the vehicle 101. In another example, the responsive data may be received and recognized by the accident assistant module via the audio system of the processing device of the driver. Without limitation, the responsive data may include audio data.

At block 230, the processor 102 controls initiation of one or more trigger events. In certain embodiments, the one or more trigger events may include providing instructions to guide the vehicle 101, requesting information from a second driver, or any combination thereof. For example, the real-time interactive assistance may be associated with any number of the one or more trigger events. Without limitation, providing instructions to guide the vehicle may include automatically maneuvering the vehicle 101 to pull it to the side of the road, such as in the case of an autonomous driving vehicle, and/or instructing the driver to pull the vehicle 101 to the side of the road. Other examples of instructions provided include driving the vehicle 101 at a predetermined speed limit, initiating vehicle brakes, and/or steering the vehicle 101 in a predetermined direction. It is understood that these instructions may be provided to an autonomous driving vehicle, which in response is configured to carry out or otherwise perform the given instructions. In other examples, it is understood that these instructions may be provided to a vehicle that is not an autonomous driving vehicle.

Without limitation, requesting information from a second driver may include requesting insurance card information from the second driver. The second driver may correspond to the driver associated with the event, such as the vehicle accident, by a second vehicle. In certain embodiments, the processor 102 may be configured to determine, based on the insurance card information, whether the insurance of the second driver has expired. To the extent that the processor determines that the insurance of the second driver has expired, the processor 102 may be further configured to take corrective action. By way of example, the corrective action may include automatically initiating and establishing communication with an emergency dispatcher and/or police and/or fire station and/or hospital. In certain embodiments, the corrective action performed by the processor 102 may be based on, for example, a location of the vehicle 101 and the second vehicle engaged in the event, and thereby determine the nearest police station, fire station, and/or hospital to automatically initiate and establish communication thereto.

It is understood that the embodiments described above is not limited to controlling initiating and establishing, by the processor 102, communication with a particular emergency dispatcher, or limited to only a second driver, or limited to only a second vehicle. Rather, any number and types of emergency dispatchers as well as any number of drivers and vehicles may be included. Further, it is understood that the drivers are not limited thereto, and that any other number and any other types of passengers may be included.

The processor 102 may be configured to determine one or more degrees of the event of the vehicle 101. Without limitation, the processor may be configured to determine a first degree of the event of the vehicle 101 and also a second degree of the event of the vehicle 101. In certain embodiments, the processor 102 may be configured to determine a first degree of the event. For example, the first degree of the event may include a minor accident associated with the vehicle 101. Without limitation, the minor accident may include a fender bender, a cracked windshield, a busted tire, a small damaged portion of an exterior of the vehicle, or any combination thereof. The minor accident may or may not be due to another vehicle. In other examples, the processor 102 may be configured to determine a second degree of the event of the vehicle 101. Further, the processor 102 may be configured to determine, based on the event comprising the minor accident, whether the vehicle 101 is driveable. For example, the processor 102 may be configured to determine, after determining the first degree of the event of the vehicle, a type of injury using one or more on-board vehicle image acquisition devices 107. By way of example, the one or more on-board vehicle image acquisition devices 107 may include any number of the interior and/or exterior cameras that may be each configured to obtain the image acquisition data and video data pertaining to the event, and in particular, the minor accident. The type of injury may include, but not be limited to, a foot injury, a head injury, or any other bodily injury relative to the driver and/or passengers of the vehicle 101. The bodily injury associated with the minor accident may be minor as compared to that of the bodily injury associated with the major accident, as further explained below.

In other examples, the processor 102 may be configured to determine if a claim, such as an insurance claim, can be made. For example, the processor 102 may be configured to establish communication with drivers and/or users of another vehicle, and thereafter receive and process payment between the parties of the vehicle and the parties of another vehicle, including exchanging information between these parties without the need for physical interaction between the parties. By way of example, the processor 102 may be configured to establish communication with a user device of the another vehicle, and thereby transmit and receive the exchanged information sufficient to process filing of a claim and/or payment between the parties. The processor 102 may be configured to determine if the insurance of another driver from another vehicle has expired and recommend processing of an insurance claim that takes into account such a consideration.

Upon determining the type of injury using the one or more on-board vehicle image acquisition devices 107, the processor 102 may be further configured to initiate and establish communication with a call center associated with the emergency dispatcher and/or the police and/or the fire station and/or the hospital.

In certain embodiments, the second degree of the event may include a major accident associated with the vehicle 101. By way of example, the major accident may be more serious and significant as compared to the minor accident of the vehicle 101. For example, the major accident may result in a substantial or totaling of the vehicle 101 and/or major bodily damage to any passengers or drivers of any number of vehicles.

The processor 102 may be configured to generate a summary of the event of the vehicle 101 based on the sensor data and the image acquisition data. For example, the summary of the event may include any number and types of reports, such as a tabularized version report, to provide vehicle trajectory data, vehicle speed data, time of day data, video data, image data, and vehicle impact data relative to time periods preceding the event, during the event, and after the event.

In certain embodiments, the driver may provide feedback to continuously improve and train the accident assistant module. For example, the driver may provide feedback, either via a request prompt or proactively, to correct or modify the instructions that it receives from the accident assistant module via the audio system. In this manner, the accident assistant module may be configured to receive the feedback, such as via the audio system or a processing device of the driver, and incorporate it to improve its instructions that it provides.

FIG. 3 depicts a schematic diagram of an example machine learning model prompt, according to one or more embodiments shown and described herein. FIG. 3 may reference and incorporate any of the above constituent components and corresponding disclosure explained above with respect to FIGS. 1 and 2, such as the example vehicle event system 100.

In certain embodiments, the accident assistant module may include one or more machine learning models that may generally comprise any type of ML model, such as a large language model (LLM) 305. Non-limiting examples of LLMs 305 include a generative pre-trained transformer (GPT), bidirectional encoder representations from transformers (BERT), XLNet, GPT-2, GPT-3, GPT-4, GPT-Neo, GPT-NeoX, GPT-J, Megatron-Turing NLG, Ernie 3.0 Titan, Claude, GLaM, Gopher, LaMDA, Chincilla, PaLM, YaLM 100B, Minerva, BLOOM, Galactica, LLaMA, Cerebras-GPT, Falcon, BloombergGPT, PanGu-Σ, OpenAssistant, PaLM 2, and others. The processor 102 may be configured to obtain, such as receive, sensor data from one or more on-board vehicle sensors 105 and/or image acquisition data on-board vehicle image acquisition devices 107. Upon obtaining the sensor data and/or the image acquisition data, the processor 102 may be configured to, via the accident assistant module, convert these types of data into text that may be used as one or more prompts 310 into a LLM 305. In certain embodiments, the one or more prompts 310 may be configured to ask the LLM 305 to produce advice, instructions, commands, requests, or any combination thereof, in the format of an audio message that may be outputted through the audio system of the vehicle. It is understood that the output, that is the request to the LLM 305 to produce the advice, the instructions, the commands, the requests, or any combinations thereof, is not limited to the audio message, and that any other media format may be utilized, including but not limited to a text message, a video message, an image message, or any combination thereof.

By way of example, a prompt 310 by the processor 102 to the LLM 305 may be: “A fender-bender occurred where there is minor damage to the second vehicle. Traffic is light on the road. Provide advice on how to handle the situation”. The determination of the “fender-bender”, its occurrence, the minor damage, and relative to the second vehicle may be determined by the processor 102 based at least on the sensor data from one or more on-board vehicle sensors 105 and/or image acquisition data on-board vehicle image acquisition devices 107. Still further, the processor 102 may be configured to determine the degree of traffic on the current road in real-time, based at least on the sensor data from one or more on-board vehicle sensors 105 and/or image acquisition data on-board vehicle image acquisition devices 107. The LLM 305 may be configured to respond, via one or more responses 320, to this prompt 310 by: “Pull your vehicle over to the side of the road and ask the other driver for their insurance. It will be okay”. In certain embodiments, rather than merely instructing the processor 102 to inform the driver to pull the vehicle to the side of the road, it is understood that for an autonomous driving vehicle, the LLM 305 may be configured to instruct the processor 102 to automatically maneuver the vehicle without driver assistance, and after determining that it is safe to do so. The driver and/or passenger of the vehicle may speak back, via the responsive data, converting the speech to text for a prompt into the LLM 305 so that an interactive and real-time conversation can be had. While FIG. 3 depicts single instances of the prompt 310 and response 320, it is understood that any number of prompts and responses may be included, and further, that the prompt 310 and response 320 may be iteratively generated and transmitted as part of the interactive and real-time conversation.

The present disclosure relates to systems and methods for vehicle events and real-time interactive assistance. Utilizing generative artificial intelligence, the systems and methods disclosed herein may be configured to generate a visualization and summary of an event, such as a vehicle accident, based on on-board sensor data and cameras, and also generate an accident assistant that speaks to a driver of the vehicle through an audio system. The generative artificial intelligence systems and methods disclosed herein may be used for personal records, for an insurance company, and/or for a government authority. To reproduce and display a visualization of the accident scene, the systems and methods disclosed herein may not only obtain data from sensors and cameras onboard a vehicle, but also from other data sources, such as the Internet of Things, in order to accurately capture the full scope of the accident. The systems and methods disclosed herein not only reproduce and the display the visualization of the accident scene, but further, the systems and methods disclosed herein interact, in real-time, with a driver or passenger of the vehicle as part of interactive assistance to determine next steps immediately following the accident, thereby improving upon user interface experience. By utilizing generative artificial intelligence to reproduce an accident scene, a realistic and informative visualization of the accident may be provided by the systems and methods disclosed herein. The generative AI model can synthesize vehicle trajectories, accurately depicting their positions, velocities, and interactions leading up to the collision. This enables the generative AI model to generate a visually compelling and informative representation of the accident, aiding in accident analysis, reconstruction, and understanding of the event. Further, the collected sensor data provides assistance to the driver in real-time in coping with the accident in an instructive and collaborative manner.

Further aspects of the disclosure are provided by the subject matter of the following clauses.

A vehicle event system, comprising: a processor; and a non-transitory, processor-readable storage medium communicatively coupled to the processor, the non-transitory, processor-readable storage medium comprising one or more instructions stored thereon that, when executed, cause the processor to: obtain sensor data and image acquisition data; generate a visualization of an event of a vehicle based on the sensor data and the image acquisition data; display, on a processing device, the visualization of the event; and transmit, via an accident assistant module, audio data to one or more passengers of the vehicle for real-time interactive assistance.

The vehicle event system of the previous clause, wherein the one or more instructions further cause the processor to generate a summary of the event based on the sensor data and the image acquisition data.

The vehicle event system of any of the previous clauses, wherein the visualization of the event further comprises vehicle trajectory data, collision dynamics data, environment data, traffic data, accident sequence over a predetermined time period, object data, and landmark data.

The vehicle event system of any of the previous clauses, wherein the processor is further configured to generate, after detection of the occurrence of the event, the accident assistant module.

The vehicle event system of any of the previous clauses, wherein the accident assistant module is configured to: transmit the audio data to a driver of the vehicle, receive, in response to the transmitted audio data, responsive data from the driver of the vehicle, and control initiation, in response to the responsive data, of one or more trigger events.

The vehicle event system of any of the previous clauses, wherein the one or more trigger events includes providing instructions to guide the vehicle, requesting information from a second driver, or any combination thereof.

The vehicle event system of any of the previous clauses, wherein the one or more instructions further cause the processor to determine a degree of the event of the vehicle.

The vehicle event system of any of the previous clauses, wherein the one or more instructions further cause the processor to: determine a first degree of the event, the first degree of the event comprising a minor accident, and determine, based on the event comprising the minor accident, whether the vehicle is driveable.

The vehicle event system of any of the previous clauses, wherein the one or more instructions further cause the processor to: determine, after determining the event of the vehicle, a type of injury using one or more on-board vehicle image acquisition devices, and initiate communication with a call center.

A method, comprising: obtaining sensor data and image acquisition data; generating a visualization of an event of a vehicle based on the sensor data and the image acquisition data; displaying, on a processing device, the visualization of the event; and transmitting, via an accident assistant module, audio data to one or more passengers of the vehicle for real-time interactive assistance.

The method of the previous clause, further comprising a summary of the event based on the sensor data and the image acquisition data.

The method of any of the previous clauses, wherein the visualization of the event further comprises vehicle trajectory data, collision dynamics data, environment data, traffic data, accident sequence over a predetermined time period, object data, and landmark data.

The method of any of the previous clauses, further comprising generating, after detection of the occurrence of the event, the accident assistant module.

The method of any of the previous clauses, wherein the accident assistant module is configured to: transmitting the audio data to a driver of the vehicle, receiving, in response to the transmitted audio data, responsive data from the driver of the vehicle, and controlling initiation, in response to the responsive data, of one or more trigger events.

The method of any of the previous clauses, wherein the one or more trigger events includes providing instructions to guide the vehicle, requesting information from a second driver, or any combination thereof.

The method of any of the previous clauses, further comprising determining a degree of the event of the vehicle.

The method of any of the previous clauses, further comprising: determining a first degree of the event, the first degree of the event comprising a minor accident, and determining, based on the event comprising the minor accident, whether the vehicle is driveable.

The method of any of the previous clauses, further comprising: determining, after determining the event of the vehicle, a type of injury using one or more on-board vehicle image acquisition devices, and initiating communication with a call center.

A non-transitory computer-readable medium comprising instructions that, when executed by at least one processor, cause the at least one processor to perform one or more operations comprising: obtaining sensor data and image acquisition data; generating a visualization of an event of a vehicle based on the sensor data and the image acquisition data; displaying, on a processing device, the visualization of the event; and transmitting, via an accident assistant module, audio data to one or more passengers of the vehicle for real-time interactive assistance.

The non-transitory computer-readable medium of the previous clause, the one or more operations further comprising: transmitting the audio data to a driver of the vehicle, receiving, in response to the transmitted audio data, responsive data from the driver of the vehicle, and controlling initiation, in response to the responsive data, of one or more trigger events, wherein the one or more trigger events includes providing instructions to guide the vehicle, requesting information from a second driver, or any combination thereof.

The preceding description is provided to enable any person skilled in the art to practice the various embodiments described herein. The examples discussed herein are not limiting of the scope, applicability, or embodiments set forth in the claims. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. Also, features described with respect to some aspects may be combined in some other aspects. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, the word “exemplary” means “serving as an example, instance, or illustration.” Any aspect described herein as “exemplary” is not necessarily to be construed as preferred or advantageous over other aspects.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c). Reference to an element in the singular is not intended to mean only one unless specifically so stated, but rather “one or more.” For example, reference to an element (e.g., “a processor,” “a memory,” etc.), unless otherwise specifically stated, should be understood to refer to one or more elements (e.g., “one or more processors,” “one or more memories,” etc.). The terms “set” and “group” are intended to include one or more elements, and may be used interchangeably with “one or more.” Where reference is made to one or more elements performing functions (e.g., steps of a method), one element may perform all functions, or more than one element may collectively perform the functions. When more than one element collectively performs the functions, each function need not be performed by each of those elements (e.g., different functions may be performed by different elements) and/or each function need not be performed in whole by only one element (e.g., different elements may perform different sub-functions of a function). Similarly, where reference is made to one or more elements configured to cause another element (e.g., an apparatus) to perform functions, one element may be configured to cause the other element to perform all functions, or more than one element may collectively be configured to cause the other element to perform the functions. Unless specifically stated otherwise, the term “some” refers to one or more.

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein include one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. §112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A vehicle event system, comprising:
a processor; and
a non-transitory, processor-readable storage medium communicatively coupled to the processor, the non-transitory, processor-readable storage medium comprising one or more instructions stored thereon that, when executed, cause the processor to:
obtain sensor data and image acquisition data;
generate a visualization of an event of a vehicle based on the sensor data and the image acquisition data;
display, on a processing device, the visualization of the event; and
transmit, via an accident assistant module, audio data to one or more passengers of the vehicle for real-time interactive assistance.

2. The vehicle event system of claim 1, wherein the one or more instructions further cause the processor to generate a summary of the event based on the sensor data and the image acquisition data.

3. The vehicle event system of claim 1, wherein the visualization of the event further comprises vehicle trajectory data, collision dynamics data, environment data, traffic data, accident sequence over a predetermined time period, object data, and landmark data.

4. The vehicle event system of claim 1, wherein the processor is further configured to generate, after detection of the occurrence of the event, the accident assistant module.

5. The vehicle event system of claim 4, wherein the accident assistant module is configured to:
transmit the audio data to a driver of the vehicle,
receive, in response to the transmitted audio data, responsive data from the driver of the vehicle, and
control initiation, in response to the responsive data, of one or more trigger events.

6. The vehicle event system of claim 5, wherein the one or more trigger events includes providing instructions to guide the vehicle, requesting information from a second driver, or any combination thereof.

7. The vehicle event system of claim 1, wherein the one or more instructions further cause the processor to determine a degree of the event of the vehicle.

8. The vehicle event system of claim 7, wherein the one or more instructions further cause the processor to:
determine a first degree of the event, the first degree of the event comprising a minor accident, and
determine, based on the event comprising the minor accident, whether the vehicle is driveable.

9. The vehicle event system of claim 7, wherein the one or more instructions further cause the processor to:
determine, after determining the event of the vehicle, a type of injury using one or more on-board vehicle image acquisition devices, and
initiate communication with a call center.

10. A method, comprising:
obtaining sensor data and image acquisition data;
generating a visualization of an event of a vehicle based on the sensor data and the image acquisition data;
displaying, on a processing device, the visualization of the event; and
transmitting, via an accident assistant module, audio data to one or more passengers of the vehicle for real-time interactive assistance.

11. The method of claim 10, further comprising generating a summary of the event based on the sensor data and the image acquisition data.

12. The method of claim 10, wherein the visualization of the event further comprises vehicle trajectory data, collision dynamics data, environment data, traffic data, accident sequence over a predetermined time period, object data, and landmark data.

13. The method of claim 10, further comprising generating, after detection of the occurrence of the event, the accident assistant module.

14. The method of claim 13, further comprising:
transmitting the audio data to a driver of the vehicle,
receiving, in response to the transmitted audio data, responsive data from the driver of the vehicle, and
controlling initiation, in response to the responsive data, of one or more trigger events.

15. The method of claim 14, wherein the one or more trigger events includes providing instructions to guide the vehicle, requesting information from a second driver, or any combination thereof.

16. The method of claim 10, further comprising determining a degree of the event of the vehicle.

17. The method of claim 16, further comprising:
determining a first degree of the event, the first degree of the event comprising a minor accident, and
determining, based on the event comprising the minor accident, whether the vehicle is driveable.

18. The method of claim 10, further comprising:
determining, after determining the event of the vehicle, a type of injury using one or more on-board vehicle image acquisition devices, and
initiating communication with a call center.

19. A non-transitory computer-readable medium comprising instructions that, when executed by at least one processor, cause the at least one processor to perform one or more operations comprising:
obtaining sensor data and image acquisition data;
generating a visualization of an event of a vehicle based on the sensor data and the image acquisition data;
displaying, on a processing device, the visualization of the event; and
transmitting, via an accident assistant module, audio data to one or more passengers of the vehicle for real-time interactive assistance.

20. The non-transitory computer-readable medium of claim 19, the one or more operations further comprising:
transmitting the audio data to a driver of the vehicle,
receiving, in response to the transmitted audio data, responsive data from the driver of the vehicle, and
controlling initiation, in response to the responsive data, of one or more trigger events, wherein the one or more trigger events includes providing instructions to guide the vehicle, requesting information from a second driver, or any combination thereof.
